(19) United States
(12) Patent Application Publication
(10) Pub. No.: US 2026/0144453 A1
(43) Pub. Date: May 28, 2026

(54) MECHANISM AND INTERFACE OF SMART INTRAORAL ACTIVE/PASSIVE SENSING

(71) Applicants: PURDUE RESEARCH FOUNDATION, West Lafayette, IN (US); University of Alabama at Birmingham, Birmingham, AL (US)

(72) Inventors: Miad Faezipour, West Lafayette, IN (US); Seyedabdollah Mirbozorgi, West Lafayette, IN (US)

(21) Appl. No.: 19/398,607

(22) Filed: Nov. 24, 2025

Related U.S. Application Data
(60) Provisional application No. 63/724,140, filed on Nov. 22, 2024.

(51) Int. Cl. A61B 5/08; A61B 5/00; A61B 5/1455; A61B 7/00

(52) U.S. Cl. CPC A61B 5/0803; A61B 5/14551; A61B 5/682; A61B 5/7267; A61B 5/7275; A61B 7/003; A61B 2503/04; A61B 2560/0462; A61B 2562/0204; A61B 2562/0219; A61B 2562/0271; A61B 2562/225

(57) ABSTRACT

An apparatus can include a mouthpiece, a sonic transmitter disposed within the mouthpiece to transmit an emitted sound signal to a respiratory system, a sonic receiver disposed within the mouthpiece to receive a reflected sound signal from the respiratory system, and neural network circuitry to execute a neural network to generate an output indicative of a health condition of the respiratory system based on the reflected sound signal.

### CROSS-REFERENCE TO RELATED APPLICATIONS

This application claims priority to U.S. Patent Application No. 63/724,140 filed Nov. 22, 2024, and entitled, “Mechanism and Interface of Smart Intraoral Active/Passive Sensing,” which is hereby incorporated by reference in its entirety.

### STATEMENT REGARDING FEDERALLY SPONSORED RESEARCH

N/A

### BACKGROUND

Respiratory diseases, a leading cause of mortality in the United States, significantly impact individuals of all ages, particularly vulnerable newborns with underdeveloped immune systems. Over 300,000 neonates face hospitalization each year due to respiratory distress syndrome caused by surfactant deficiency. These patients have a heighted risk of developing chronic lung diseases, severe infections, and long-term cognitive impairments. The absence of suitable pulmonary monitoring technologies tailored to neonatal needs poses a significant challenge, with a $7 billion cost burden over a decade in neonatal respiratory care. Assessing pulmonary health on infants is complex due to their delicate physiologies and inability to follow instructions or control breathing.

### SUMMARY

Aspects of the present technology may include mechanisms of active sensing and smart pacifier/wearable circuit interfaces, that may seamlessly be integrated with smartphones (or other computing devices, such as, for example, a computer, laptop, tablet, etc.) for accurate, reliable, and non-invasive personalized pulmonary assessment through characterizing reflected breathing sounds using advanced signal processing and AI-guided machine learning/deep learning (ML/DL) models.

Example aspects of the present technology may include three mechanisms for non-invasive respiratory profiling: (1) Average cardiorespiratory profiling, by passively sensing of breathing sounds, (2) Real-time cardiorespiratory profiling, by active sensing of breathing sounds, and (3) Continuous cardiorespiratory profiling by smart circuit interfaces.

Some embodiments of the disclosure provide an apparatus. The apparatus can include a mouthpiece, a sonic transmitter disposed within the mouthpiece to transmit an emitted sound signal to a respiratory system, and a sonic receiver disposed within the mouthpiece to receive a reflected sound signal from the respiratory system. The apparatus can include a neural network circuitry to execute a neural network to generate an output indicative of a health condition of the respiratory system based on the reflected sound signal.

Some embodiments of the disclosure provide an apparatus. The apparatus can include a mouthpiece, one or more acoustic transducers coupled to the mouthpiece, and a processor in communication with the one or more acoustic transducers. The processor can be configured to receive, from the one or more acoustic transducers, a sound signal from a respiratory system, provide the sound signal to a trained model that identifies respiratory health conditions, and receive, from the trained model, an output indicative of a respiratory health condition.

Some embodiments of the disclosure provide an apparatus. The apparatus can include a mouthpiece, one or more acoustic transducers coupled to the mouthpiece, and a processor in communication with the one or more acoustic transducers. The processor can be configured to receive, from the one or more acoustic transducers, a sound signal from a respiratory system, provide the sound signal to a trained model that identifies respiratory health parameters, and receive, from the trained model, an output indicative of a respiratory health parameter.

Some embodiments of the disclosure provide a method. The method can include receiving, using one or more computing devices, a sound signal, the sound signal being from a respiratory system of a subject, providing, using the one or more computing devices, the sound signal to a trained model that identifies a respiratory health condition or a respiratory parameter, and receiving, using the one or more computing devices, an output from the trained model indicative of the respiratory health condition or the respiratory parameter.

Some embodiments of the disclosure provide a method. The method can include emitting a first sound signal into a respiratory system of a subject, receiving a second sound signal from the respiratory system of the subject, the second sound signal being an echo in response to the first sound signal, receiving a third sound signal from the respiratory system of the subject, the third sound signal being a passive sound signal acquired in the absence of a stimulus sound signal provided to the respiratory system, providing the second sound signal and the third sound signal to a trained model that identifies the respiratory health condition or the respiratory parameter, and receiving an output from the trained model indicative of the respiratory health condition or the respiratory parameter.

### BRIEF DESCRIPTION OF THE DRAWINGS

FIGS. 1A and 1B illustrate various aspects of the present technology.

FIG. 1C illustrates example breathing sound waveforms comparing a healthy subject to a subject having chronic obstructive pulmonary disease (COPD).

FIGS. 1D and 1E illustrate example spectrograms corresponding to the breathing sound waveforms of FIG. 1C.

FIGS. 2A-2D illustrate example results of breathing sound signals and associated spectrograms.

FIGS. 2E and 2F illustrates an example COMSOL lung model and simulating sound response to an active sound stimulus.

FIG. 3 illustrates an example neural network for respiratory function monitoring.

FIG. 4 illustrates another example neural network for respiratory function monitoring.

FIG. 5 illustrates example frequency responses to different active sound stimuli frequencies in determination of respiratory conditions.

FIGS. 6A and 6B illustrate an example wearable device to provide respiratory function monitoring.

FIG. 7 is an example flowchart for performing respiratory function monitoring using a neural network.

FIG. 8 illustrates an example of training a neural network or other suitable machine learning model to provide respiratory function monitoring.

FIG. 9 is a block diagram of an example respiratory function monitoring system.

FIG. 10 is a block diagram of example components that can implement the system of FIG. 9.

### DETAILED DESCRIPTION

Reliable, precise, and neonate-friendly monitoring mechanisms capable of accurately tracking respiratory metrics are crucial for managing in and post-hospitalization care, especially for high-risk infants. The challenges and gaps in current monitoring capabilities (lack of real-time, continuous, accurate and compatible monitoring), highlight the need for innovative and suitable solutions for infants under the age of three. For example, typical approaches involve the use of spirometry to obtain respiratory parameters and inform clinical decisions. However, spirometry involves forced beathing from a user—and at times, requires the maximum possible expelling effort from a user to obtain some of these parameters. Obviously, these forced air approaches can be difficult, if not impossible, to perform, particularly for high-risk infants (e.g., infants may not be able to forcibly expel out, and other children may not be permitted by a practitioner from doing so). The techniques described herein permit detection of respiratory parameters in a continuous manner, while a subject breathes normally (e.g., without any further expended effort). Therefore, the techniques herein allow for spirometry of neonates—including detecting respiratory parameters and clinical respiratory condition diagnoses.

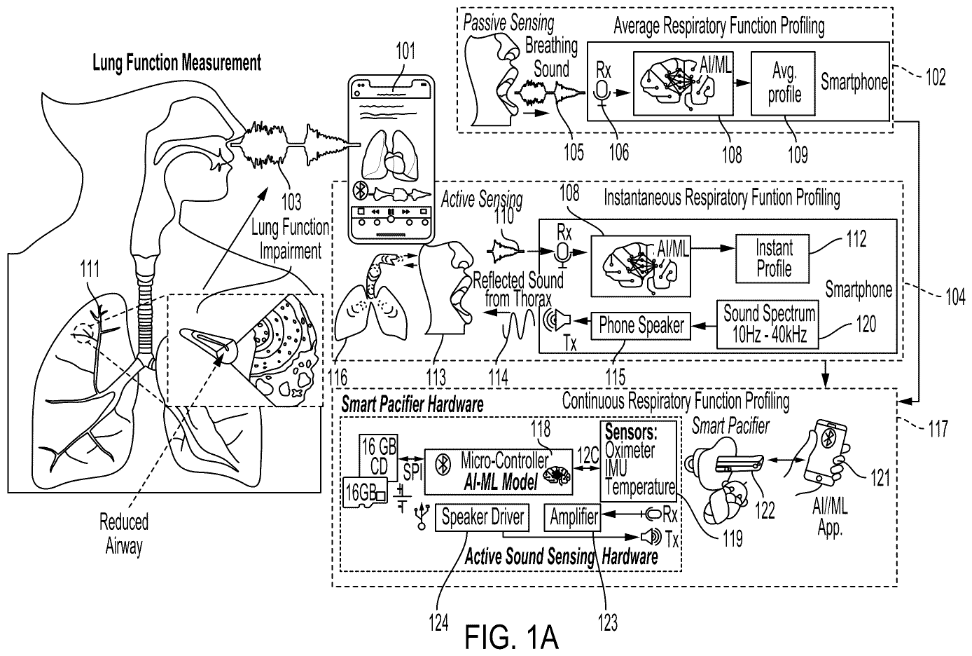

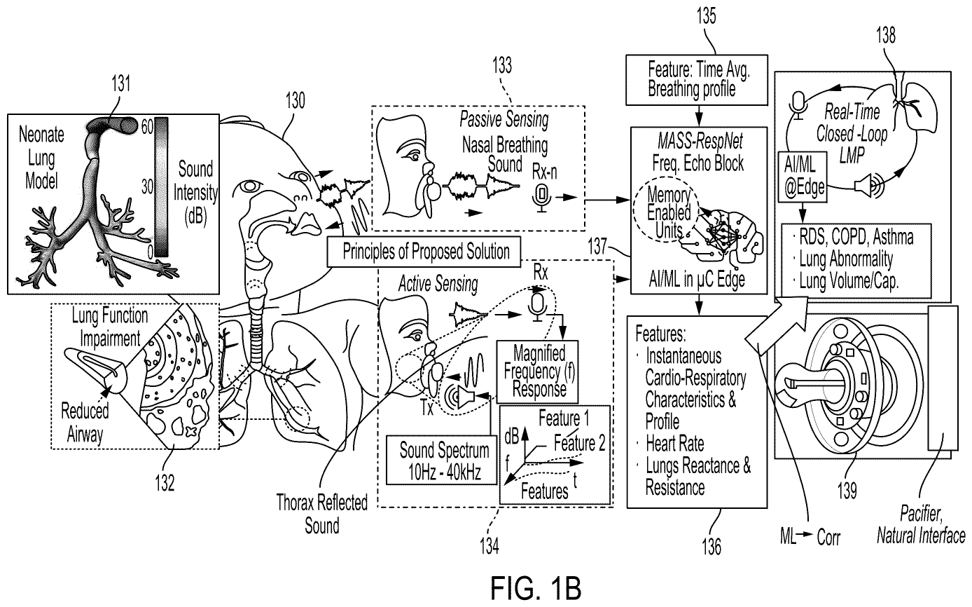

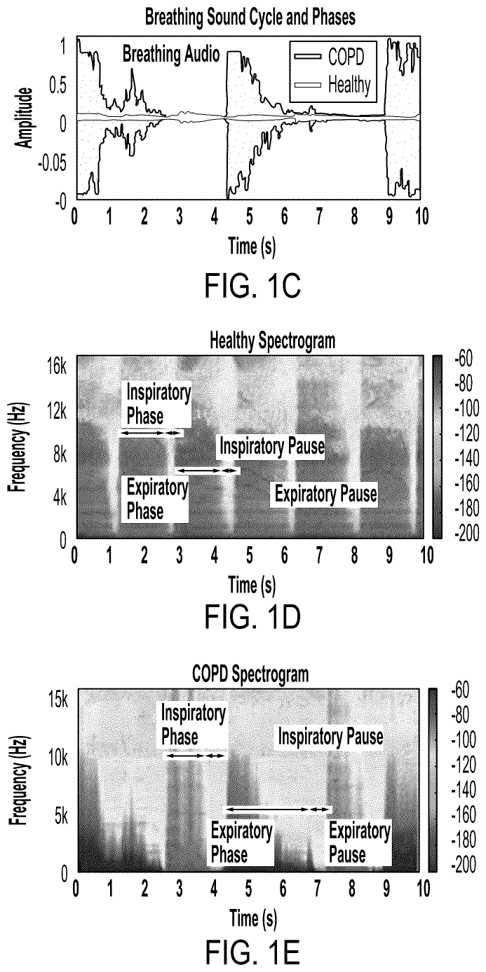

FIG. 1A illustrates various aspects of the present technology. In some implementations, a mobile device 101 may capture a sound signal from a subject's respiratory system 111. In some implementations, the system may provide an active sound sensing approach 104 that involves exposing the intended target area 111 to a stimulus sound 114 and analyzing the response (reflection) 110 in terms of sound waves. In some examples, the stimulus sound 114 targets the respiratory function 111 intraorally. As the stimulus sound travels through the airway into the lungs, it encounters the tissues and organs along the way, whose reflection when bouncing back will produce a sound in response (echo) while breathing. For example, reflected breathing sounds 110 sensed through the thorax area and exiting from the mouth or nose may be referred to as intraoral or nasal sound sensing approaches. Active sound sensing 104 may comprise capturing reflected sounds 110 in the presence of a sound stimulus 114. Aspects of the present technology may further provide a passive sensing approach 102 involving capturing breathing sounds 105 in an absence of external sound stimuli. In some examples, a device 101 may include a speaker 115 that may be used to play the sound stimuli 114 and a microphone 106 may be used to sense these sounds. In some implementations, the active sound signal may be emitted while the sound receiver receives the reflected signal (e.g., they may overlap in time). In further implementations, the emitted sound signal may complete before receiving the reflected sound signal, for example, via a pulse, chirp, etc.

In some implementations, sound stimuli 114 may be provided at multiple frequencies. For examples, sound stimuli may be provided as frequency sweeps. For example, sound stimuli 114 may be provided at infrasound frequencies, audible frequencies, or ultrasound frequencies, such as in the range of 10 Hz to 40 kHz. In some examples, the sound response to the sound signal may be captured while a subject is breathing. This response may be analyzed via various techniques. In some implementations, different frequencies of the stimulus sound may be employed in the active sensing approach. This may reveal different cardiorespiratory characteristics, as certain frequencies can resonate with the reflected response that are dominant for certain cardiorespiratory features. More specifically, a certain frequency may more accurately predict the lung volume measure, or breathing rate, while another frequency may more accurately identify a pulmonary disease, infection, airway blockage or lung resistance. In various implementations, frequency analysis may be performed on reflected sound signals over different frequencies of stimuli. Different signals in terms of characteristics such as magnitude and phase to identify aspects such as resonances, constructive/destructive interference, filtering, or modulation effects the stimulus sound may have on reflected sound signals. Various implementations may utilize any suitable sound stimulus profile, such as sinusoidal tones, pulses, etc.

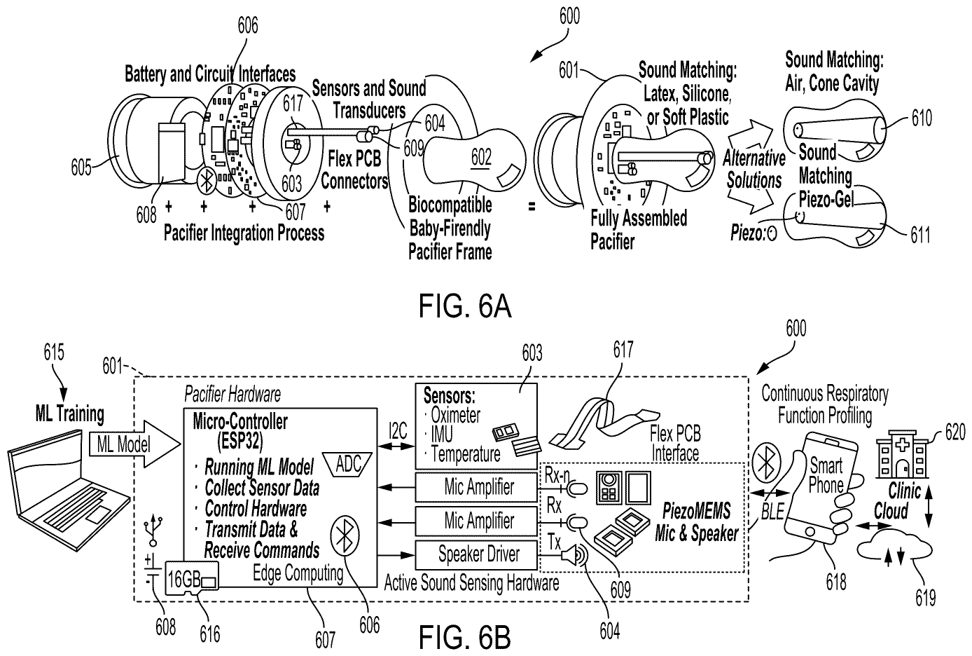

FIG. 1B illustrates further aspects of the present technology, and in particular with respect to a neonate subject 130. As discussed below, in some examples, passive sound sensing 133 is performed to capture breathing sounds and active sound sensing 134 is performed to capture reflected breathing sounds resulting from sound stimuli applied while a subject breathes. AI/ML processes 138 may be applied to combine active sound sensing 134 with artificial intelligence (AI)-based correlations 137 through multimodal active sound sensing of respiratory characteristics 136. In some implementations, wearable circuit interfaces 139 may be provided, such as suitable for integration within pacifier-like structures (see, e.g., FIGS. 6A-6B). The present technology may provide precise real-time continuous pulmonary function monitoring through a closed-loop Lung-function Monitoring Pacifier (LMP) framework 138, addressing a critical need for diverse patient groups, especially neonates. In some examples, reflected intraoral sounds may be correlated to generate time-averaged breathing profiles in the passive mode, while accurately extracting instantaneous cardio-respiratory metrics in the active mode with sound stimuli present. In some cases, different active sound frequencies may be applied to reveal different pulmonary characteristics. The sound sensing mechanism may be integrated into a pacifier 139 (a natural interface for babies) for continuous LMP functioning. In some examples, a sound stimulus may include a plurality of tones. For instance, a plurality of tones may be emitted as a multi-tone sonic signal, a series of temporally separated tones, combinations thereof, etc.

FIGS. 1C-1E illustrate example sound signals that may be captured from a subject and example spectrograms of such signals. Respiration, essential for living organisms, involves oxygen intake and carbon dioxide release through breathing. Respiratory (breathing) sounds, reflecting the four airflow phases of inhalation, inhalation pause, exhalation, and exhalation pause (FIG. 1C) may be used to diagnose breathing conditions. For example, the Computer Respiratory Sound Analysis (CORSA) society has defined over 162 terms categorizing normal and adventitious sounds. Adventitious sounds like crackles, wheezes, coughs, snoring, rhonchus, squawk, and stridor possess distinct frequency characteristics in different inhale/exhale phases, aiding accurate respiratory diagnosis. Adventitious sounds are often associated with lung related complications such as bronchopulmonary dysplasia (BPD), respiratory distress syndrome (RDS), chronic obstructive pulmonary disease (COPD), pneumonia, pulmonary edema, tracheal stenosis, asthma, bronchitis, apnea, post prematurity respiratory disease (PRD), etc. For example, FIG. 1D illustrates an example spectrogram of a healthy respiratory sound signal while FIG. 1E illustrates an example COPD spectrogram, showing some example differences between a healthy and unhealthy respiratory system.

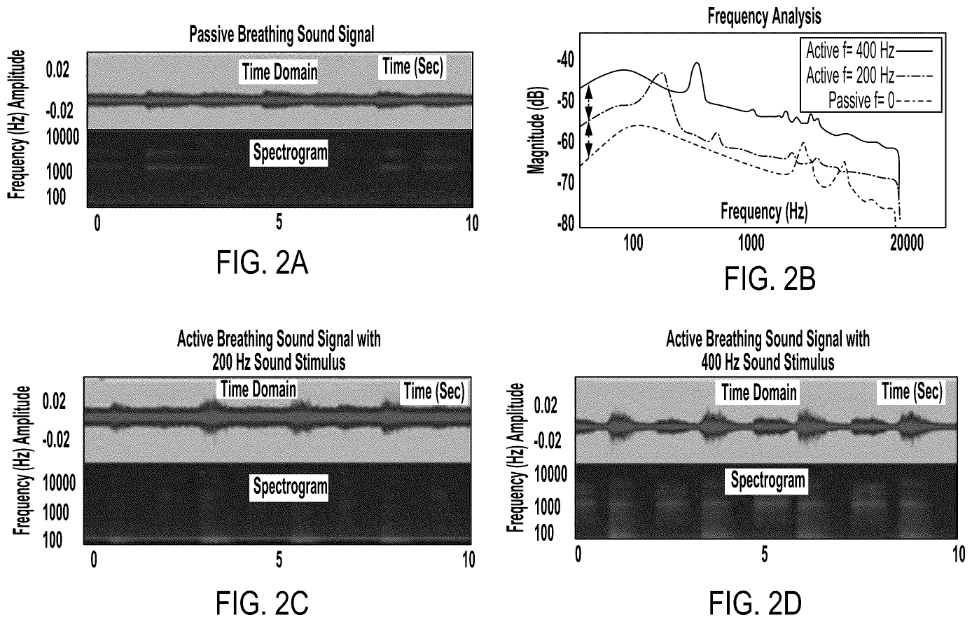

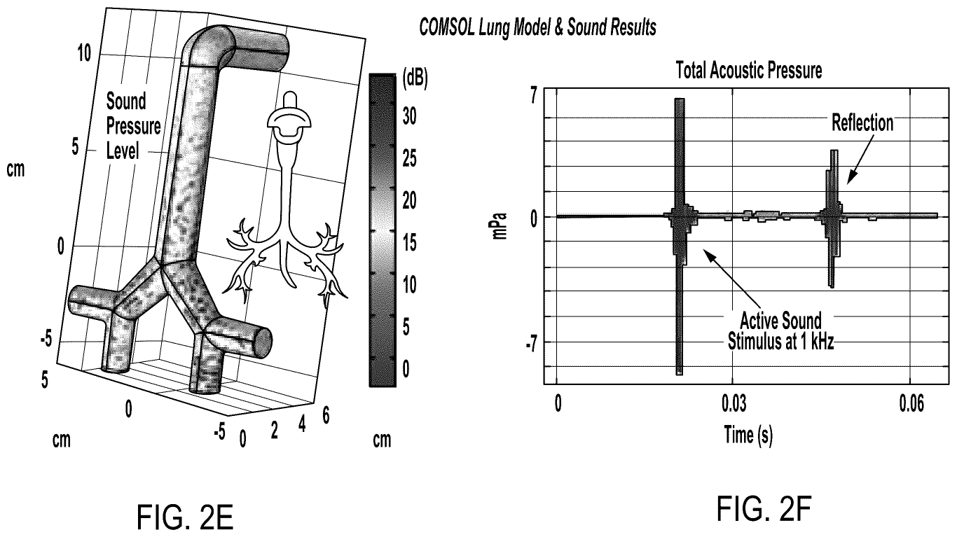

FIGS. 2A-2D illustrate results of an example experiment to generate sound waves and record the breathing sound response using a smartphone (speaker and microphone), simultaneously. In this example experiment a tone generator smartphone application was used to play a stimulus sound at a user-provided frequency through the speaker near the mouth, and the reflected sound was captured through the microphone. FIG. 2A illustrates an example of passive breathing sound capture (e.g., breathing sound without an active sound stimulus). For ease of explanation, passive sound capture may be described as a 0 Hz active sound stimulus. FIG. 2B illustrates profiles providing analyses of different sound stimuli (including passive sound). FIG. 2C illustrates an example time domain waveform and spectrogram for a reflected sound signal comprising breathing sounds recorded in the presence of a 200 Hz sound stimulus. FIG. 2D illustrates an example time domain waveform and spectrogram for a reflected sound signal comprising breathing sounds recorded in the presence of a 400 Hz sound stimulus. As illustrated, in this experiment, the active sensing approach displayed more prominent (sharper) features of the signal in the time domain, frequency spectrogram, and frequency analysis.

FIGS. 2E-2F illustrate results of preliminary simulations performed via COMSOL ultrasound simulations for a lung model (FIG. 2E) of the airway passage (trachea) and a few branches representing the bronchioles and alveolar capillary of the two lungs. The model was designed with geometry and material (impedance) resembling human lungs for babies (more delicate, shorter trachea). FIG. 2F presents the sound pressure intensity results for 1 kHz sound stimulus reflected along the passage, confirming producing safe sound levels (<60 dB) that can be correlated with pulmonary characteristics.

In some implementations, captured sound signals may be preprocessed, such as via segmentation into smaller units for processing (e.g., 20 seconds), denoising, padding, segmentation, normalization, deriving frequency spectrograms, etc.

Some implementations may comprise a multi-modal configuration comprising both active and passive sound sensing modes 102, 104. For instance, a multi-modal implementation may balance performance and battery/low power considerations. For example, in some implementations the active sound sensing approach (active sound stimuli) need not be running continuously. In some cases, passive sounds can be sensed through the microphone continuously to provide an average profile 109. In some implementations, the average profile generated via passive sound capture may be calibrated or updated via processing results of active sound capture modes (e.g., to compensate for passive reflected breathing sounds averaged over time not carrying dominant frequency responses at the immediate time a physiological activity or event is taking place). In some cases, the active sound sensing approach through playing the stimulus sounds may provide a real-time profile 112, in some cases, in conjunction with the average profile 109. In some implementations, the passive sensing approach 102 may provide an average profile 109 (over a longer period such as days, weeks, or months) of the measure under investigation (e.g. lung volume, heart rate). In some cases, the active sensing approach 104 may support constructive interference magnifying the frequency response in real-time and may thus enable more accurate immediate observations (e.g., real-time output for sounds captured during an active stimulus session).

Aspects of the present technology may provide an artificial intelligence/machine learning system 108 to process audio signals to create breathing profiles 109, 112 (e.g., predict cardiorespiratory features or abnormalities). In some implementations, multi-modal active sound sensing of respiratory features may be determined via a neural network 108, such as a convolutional neural network (CNN), for profiling breathing with classification (healthy or not healthy) or regression outputs pertaining to FVC, FEV1/FVC, SPO2, and VO2Max, incorporating skip connections and attention mechanisms.

In some examples, a multimodal configuration may be implemented to support both active and passive modes of sound sensing, for instance to balance performance and manage battery life. For instance, active sound sensing, which involves generating sound stimuli, need not be running continuously. In some examples, passive sound sensing may operate continuously via the microphone to develop an average profile, calibrated regularly using active sound stimuli. The active sensing approach, on the other hand, allows for constructive interference magnifying the frequency response in real-time, and may thus enable more accurate immediate (real-time/instantaneous) observations.

In various examples, the control software may trigger active sensing periodically or when an abnormal event is detected, following a closed-loop design for efficient management of sound stimulus generation and processing. It may use reflected sounds as input to generate spectrograms for the subsequent AI-based data processing module and may generate or halts stimuli based on feedback from the subsequent AI module (detected events) or user input. As discussed elsewhere, various frequencies may be used for health determination, for example, via frequency range sweeps between 10 Hz-40 kHz. In some examples, particular stimulus sound signal/frequency whose reflection results in the most distinct correlation with the cardiorespiratory measures may be deployed.

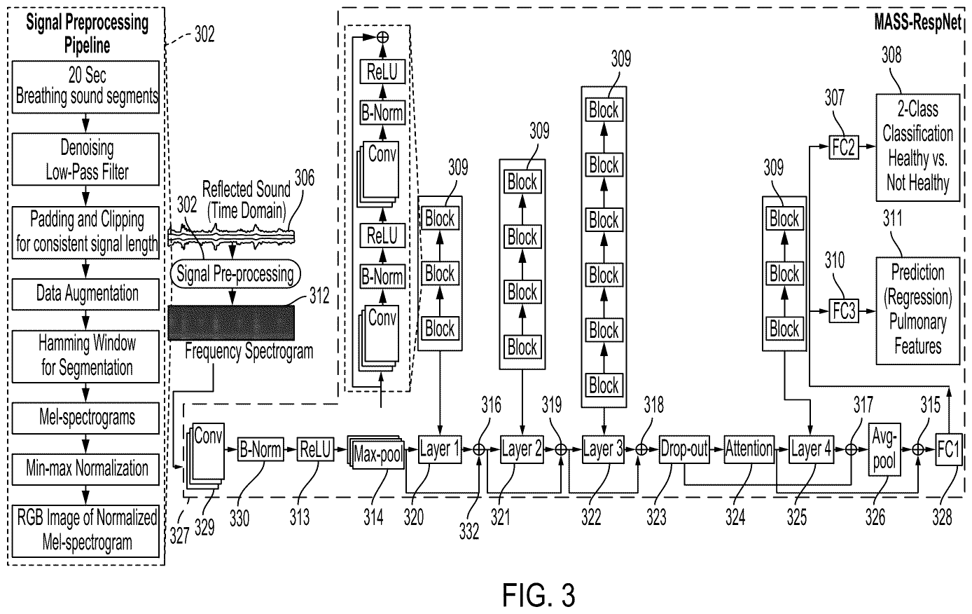

FIG. 3 illustrates an example AI/ML system for respiratory sound signal processing. Of course, the illustrated neural network example is one example of various potential network architectures. Aspects of the present technology may be implemented via any suitable neural network architecture. For example, as described below, the AI/ML model and architecture uses a multi-modal approach where both active sensing (e.g., sound received from echoes, or in response to a probing sound signal) are provided to the model, which is fine-tuned to extract features from the respiratory sound spectrogram (e.g., from both the active and passive spectrogram). This multi-modal sound sensing can be enabled by having dedicated memory enabled echo blocks.

In this example, a sound signal 306 may be captured from a subject's respiratory system. In some cases, sound signal 306 may be processed via a preprocessing pipeline 302. Pipeline 302 may comprise various processing operations, such as chunking operations (e.g., dividing a captured signal into equal-duration segments (e.g., 10, 20, 30 seconds, etc.). Auditory processing operations such as denoising and low-pass filtering operations may be applied to the sound signal (e.g., to the segments). Padding/clipping operations may be applied after denoising/filtering to generate consistent signal segments. Data augmentation operations may be applied, such as to change speed/pitch characteristics, time/frequency masking, polarity inversions, temporal stretching, phase modification, etc. Window processing, such as Hamming window segmentation may be applied to concentrate spectral energy (e.g., avoid signal edge effects). Signal preprocessing may further comprise generating a spectrogram, such as a mel-spectrogram (e.g. Fourier transforming and non-linear conversion to mel units). In some cases, spectrograms may be normalized, such as via min-max normalization, etc. In some examples, a neural network may operate on image data, and preprocessing may comprise generating an image 312 (e.g., RGB color image data) of the spectrogram (e.g., multi-channel 2D data representative of a spectrogram).

In some implementations, a spectrogram 312 may be processed via a neural network 327 to generate a classification 308 or predication of pulmonary features (e.g., regression). In the illustrated example 327, spectrogram 312 may be processed via various subnetworks. For example, an initial subnetwork may comprise a convolutional layer 329, followed by a batch normalization 313, activation layer 313, such as a rectified linear unit (ReLU) and a pooling layer 314, such as a max-pool layer 314.

In the illustrated example, neural network 327 may comprise a plurality of subnetwork layers 320, 321, 322, 325. For example, layers 320, 321, 322, 325 may comprise a plurality of neural network blocks 309, where each block may comprise one or more sets of convolutional blocks, batch normalization blocks, ReLU blocks. In some cases, the output of each block is combined (e.g., dilated), the result of which is input to a next block. As illustrated, in some implementations, different layers 320, 321, 322, 325 may comprise different numbers of blocks 309. In this example, the output of each layer is dilated 316, 319, 318, 317 with the input of the layer via skip connections 332. For instance, skip connections 332 may avoid issues such as vanishing gradient. Additional neural network components may be included between layers 320, 321, 322, 325. For instance, drop-out layers 323 and attention layers 324 may be included (e.g., prior to last layer 325). In this example, the outputs of the drop out 323 may be dilated 317 with the output of last layer 325. Further processing blocks, such as an average pooling layer 326 may be applied, which may be dilated 315 with a prior output, such as the output of attention layer 324. The final layers 328, 307, 310 of the network may comprise fully connected network layers and may generate a classification 308 and predictions 311 of various respiratory parameters such as forced vital capacity (FVC), FEV1/FVC (e.g., a ratio of forced expiratory volume in the first second (FEV1) to FVC)), oxygen saturation (SPO2), heart rate (HR), breathing rate (BR), maximal oxygen consumption (VO2Max), etc. For instance, a first fully connected layer 328 may generate an output of features that are input to a fully connected classifier 307 or a fully connected regression layer 310. In this example, network 327 is provided with RGB image data presenting the mel-spectrogram of 20 second segments of the reflected breathing sound at the output of the signal pre-processing pipeline 302, and generates predictions 308, 311 for the aforementioned four cardiorespiratory parameters, to provide a breathing profile (e.g., profile 110). As an example, classification 308 may provide an indication of a health status of a subject and regression 311 may provide data indicative of the subject's respiratory system (e.g., for the creation of a “digital twin” model of the subject's respiratory system).

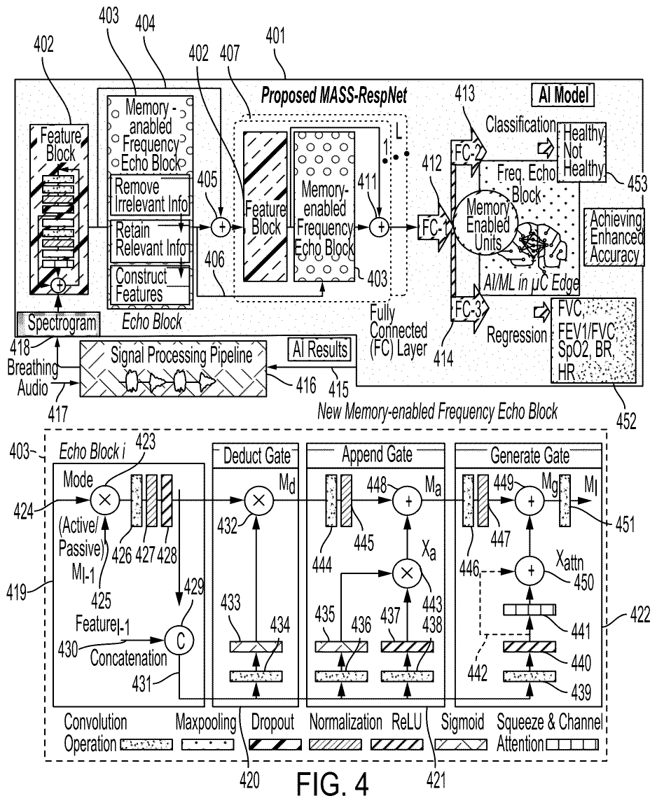

FIG. 4 illustrates another example AI/ML system for respiratory sound signal processing. For example, as indicated above, system 401 may correlate the active/passive reflected breathing sounds with measurable pulmonary features through an AI-guided pipeline. For example, system 401 may be implemented in a network-connected mobile medical device (e.g., an Internet-of-Medical-Things (IoMT) device), such as an IoMT pacifier system. Thus, system 401 may be an example of an integration of AI at the edge, including an AI/ML system for audio signal processing that may create breathing profiles (e.g., predict cardiorespiratory features or abnormalities) for enhancing pulmonary assessment in real-time. Aspects of the present technology may further include IoMT software/hardware integration of breathing sound reflection analysis on a pacifier MCU, which may be linked with a smartphone (or other computing devices, such as, for example, a computer, laptop, tablet, etc.).

In the illustrated example, neural network 401 operates on a spectrogram 417, such as an RGB spectrogram (e.g., color may indicate spectral power, etc.). In some examples, a captured sound signal 417 (e.g., reflected sound signal or passive sound signal) may be processed via a signal processing pipeline 416. For instance, pipeline 416 may be implemented as described with respect to pipeline 302. For instance, a sound signal 417 may be segmented into chunks for processing (e.g., 20 seconds or smaller), followed by various processing operations, such as denoising, padding, segmentation, normalization, etc. The processed sound signal may be transformed to a frequency spectrogram 418 (e.g., using a Fast Fourier transform (FFT)). In some implementations, a feedback signal 415 may be provided by neural network 401 to affect the pipeline operations 416. For instance, feedback signal 415 may be used to control parameters (e.g., normalization values, denoising parameters, etc.), to control which operations are performed on the input signal 417 (e.g., various data augmentation operations, etc.), etc. As another example, feedback signal 415 may comprise signals determined from an active sound operation for calibration of a passive sound operation.

In some implementations, neural network 401 may comprise one or more feature extraction neural networks 402, 408 (“feature block 402, 408”). A feature block 402, 408 may extract local and global features from audio spectrograms 418 using dilated convolution layers to expand receptive fields and identify important input regions. Self-attention mechanisms may focus on significant areas, while dropout may prevent overfitting. In the illustrated example, neural network 401 comprises an input feature extraction block 402. For example, feature block 402 may comprise a dilation CNN comprising one or more sets of convolution layers, normalization layers, ReLU layers, dropout layers, pooling layers, attention layers (e.g., squeeze and channel activation), etc. For instance, feature block 402 may comprise a first subnetwork comprising set of layers comprising (processing order from top down) a convolutional layer, a batch normalization layer, a ReLU layer, and a dropout layer. In this example, the output of this first subnetwork is dilated with an output of a second subnetwork via a skip connection. Continuing the example, the second subnetwork may comprise a convolution layer, batch normalization layer, ReLU layer, max pooling layer, and a squeeze and channel activate layer. The output of the second subnetwork (e.g., extracted features with attention) may be dilated with the spectrogram and output to further network components.

Neural network 401 may further comprise one or more feature generation subnetworks 403 (“memory enabled frequency echo blocks” or “echo blocks” 403). Echo blocks 403 may manage memory content, such as by discarding irrelevant data, retaining relevant data, and constructing effective features. For example, echo block 403 may capture active sound sensing features and enhance spectral features of breathing audio by capturing and refining respiratory audio echoes from reflected signals. For example, echo block 403 may operate in an active audio signal mode and be deactivated in a passive signal mode. Accordingly, an echo block 403 may support network 401 selectively considering relevant information pertaining to constructive frequency modulation and magnification through spatial and channel attention in the echo block, for active sensing and real-time/instantaneous profiling.

For instance, an input of echo block 403 may comprise an active/passive select input 424. For example, in a passive signal mode, neural network 401 may apply the feature blocks 402, 408 while skipping echo blocks 403. For instance, an echo block may comprise a mode select input 424 that is applied to control whether the echo block is applied to an input. In this example, mode select input 424 may be a constant ‘1’ for an active signal mode and a constant ‘0’ for a passive signal mode. In this example, input 425 and mode select 424 may be combined (e.g., via a tensor product 423) such that the input is nulled in the passive signal mode.

As described below, attention may be applied to high-level features before fully connected (FC) linear layers. Skip connections link outputs across layers, which may mitigate vanishing gradient issues. As illustrated, neural network 401 may comprise an initial layer (e.g., I=0) layer comprising a first feature block 402 and a first echo block 403. The neural network 401 may further comprise a plurality of L layers 407. These layers and subnetworks may be connected via various skip connections 404, 406 and dilation combinations 405, 411 as illustrated. The L layers, each containing 2×i feature and echo blocks (1≤i≤L) may enhance feature generation before the FC layers 412, 413, 414.

For layers 407, the output M_{I} of a layer may be passed to a next layer as input 425 M_{I-1}. Input 425 may be processed via various layers, such as a convolutional layer 426, a batch normalization layer 427, and a ReLU layer 428. The output of ReLU layer 428 may be concatenated 429 with a preceding feature output 430 to produce a first input 431. Echo blocks 403 may include a feature input 430 to receive the output of previous feature block 402. Here, the input may be processed via input 431 by a data removal subnetwork 420 (“deduct gate” 420), which may comprise a convolution layer 434 followed by a bounding layer 433 (e.g., a sigmoid layer). The output of bounding layer 433 may be combined 432 (e.g., via a tensor or Kronecker product) to produce a deducted output M_{d}. M_{d} may be input to a data retrieval subnetwork 421 (“append gate” 421).

Append gate 421 may receive the input 431 and apply parallel operations, such as a first branch including a convolution layer 436 and bounding layer 435. A second branch may include a convolutional layer 436 and a ReLU layer 437. The output of both branches may be combined 443 (e.g., via a tensor product), to provide an output Xₐ. In this example, the output M_{d} of deduct gate 420 may be processed via neural network layers, such as a convolutional layer 444 and a ReLU layer 445 and then dilated 448 with Xₐ to produce an output Mₐ.

A feature generation subnetwork 422 (“generate gate” 422) may be applied to input 431 and append gate output M_{A}. In this example, generate gate 422 may comprise a convolutional layer 439 followed by a ReLU layer 440. The output of ReLU layer 441 may be passed to an attention layer 441 (e.g., a squeeze and channel activation layer 441) and passed via a skip connection 442. Here, the output of attention layer 441 and ReLU layer may be dilated 450 to produce an output attention vector Xₐₜₜₙ. Append gate 421 output Mₐ may be input to generate gate 422 and processed via neural network layers, such as a convolutional layer 446 followed by a ReLU layer 447. The output of ReLU layer 447 may be dilated 449 with Xₐₜₜₙ to produce an output M_{g}, which may be processed via a convolutional layer 451 to produce an output M_{I}.

The output of layers 407 (e.g., M_{L}) may be passed to one or more output layers, such as fully connected layers 412, 413, 414. The final layers 412, 413, 414 of the network 401 may comprise fully connected networks and may generate a classification 453 and predictions 452 of conditions such as FVC, FEV1/FVC, SPO2, HR, BR, VO2Max, etc. For instance, a first fully connected layer 412 may generate an output of features to be input to a fully connected classifier 413 or a fully connected regression layer 414. As an example, classification 453 may provide an indication of a health status of a subject and regression 452 may provide data indicative of the subject's respiratory system (e.g., for the creation of a “digital twin” model of the subject's respiratory system).

In some implementations, neural network 401 may combine breathing audio spectral analysis as 2D images with memory-enabled echo units and feature blocks. While described with respect to respiratory health, neural network 401 may be adapted to a wide range of audio, acoustic, and image analysis tasks (such as medical image segmentation).

### Experiments

Various examples conducted in support of the present technology are described below.

In a first experiment, PyTorch was used to generate a neural network (MASS-RespNet) model as described with respect to FIG. 3 on Purdue University's Gilberth computing cluster, equipped with NVIDIA A30 Tensor GPUs, a single node with 40 cores and 72 GB memory. Ground truth values were obtained from Spirobank II® MIR Spirometer for lung volumes, a pulse oximeter for SpO2, and (maximum and resting) heart rate values for VO2Max.

In an experiment with 12 subjects (6 healthy, 6 COPD), passive sensing was used on 2-minute breathing recordings to classify healthy and COPD signals, differentiate inhales/exhales, and determine respiratory rates. With an 80% training and 20% testing split, the model achieved 85-87% accuracy classifying COPD and healthy individuals using 20-second segments and 100% accuracy considering entire audio recordings per subject. The breathing cycles and phases have also been detected accurately.

In another experiment, the active sensing approach has been tested using the MASS-RespNet model on 24 healthy control and test subjects with 3 minutes of breathing sounds and the 80%/20% data split. The Mean Squared Error (MSE) loss predicting the cardiorespiratory measures, was 0.0074 in the passive sensing approach, and reduced to 0.0007 with the active sensing approach. This order of magnitude improvement with the active sensing approach may illustrate that frequency modulation contributes to achieving improved results for breathing sound analysis. Additionally, the effectiveness of active sensing was investigated at different frequencies. Here, it was observed that f_{act}=400 Hz outperforms the other frequencies (results in lower loss).

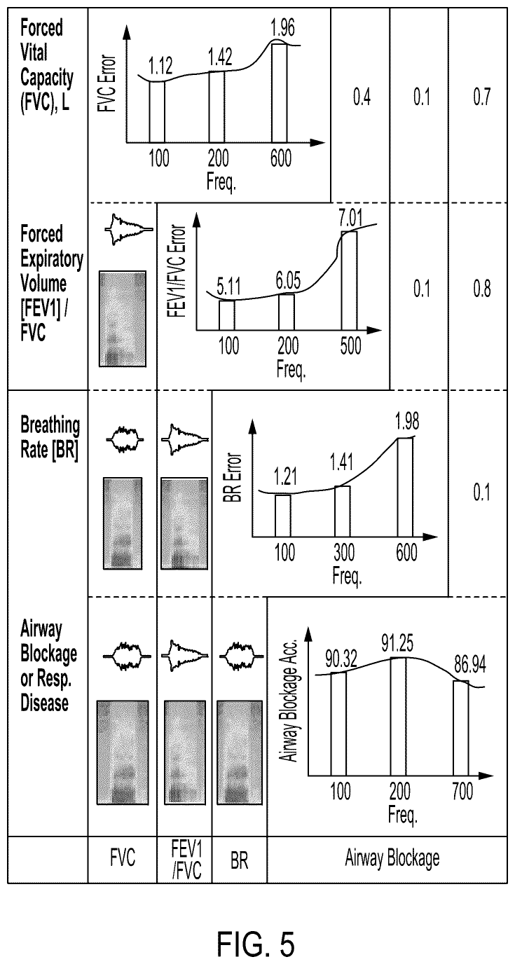

In another experiment, active and passive sensing was applied using MASS-RespNet as illustrated in FIG. 4 on 27 healthy and patient subjects (with COPD, Asthma, Shortness of Breath, or Diabetes) with 3 minutes of breathing sounds and the 80%/20% data split. The classification accuracy (healthy vs. COPD) increased from 86% in passive sensing to 95% in active sensing. Particularly, the Mean Squared Error (MSE) loss predicting the cardiorespiratory measures in the active mode was reduced from the passive mode, with an order of magnitude improvement (10 dB) in the active sensing approach, indicating that frequency modulation is substantial in achieving better results for breathing sound analysis. Different active sound frequencies between 100 Hz and 700 Hz were applied, with different frequencies resulting in lower error or higher accuracies for different pulmonary characteristics. Preliminary data with active sound resonance at different frequencies is depicted in FIG. 5.

### Example Implementation

FIGS. 6A and 6B illustrate an example system 600 including a wearable intraoral device 601 for respiratory health monitoring. As a particular example, device 601 may comprise a device including a mouthpiece to provide a pathway for intraoral sound transmission/reception. For instance, device 601 may comprise a pacifier or other device suitable for use by a neonate subject. For example, system 600 may provide real-time analysis of a subject's respiratory condition. For instance, system 600 may detect issues that may occur during sleeping, such as sleep apnea. As another example, system 600 may employed in neonatal intensive care units where rapid detection of breathing issues is especially critical for positive health outcomes. As a particular example, a system provided as a smart pacifier may provide a natural and comfortable interface for neonates, supporting long-term or continuous health monitoring.

In some examples, device 601 may include a mouthpiece 602 for insertion into a subject's mouth to provide a pathway for intraoral sound transmission/reception. For instance, in the illustrated example of a pacifier device 601, pacifier 601 may include a housing comprising a pacifier tip (“nipple”) 602. In various implementations, the mouthpiece 602 can be a nipple and may comprise materials that provide sound transmission/reception characteristics while being suitable for use by a neonate. For instance, nipple may comprise a rubber or other polymer materials such as latex, silicone, plastic, etc. For example, nipple may comprise a material having an index of acoustic refraction suitable for transmission and reception of various frequencies. For instance, as described above, some implementations may utilize sounds in multiple domains, such as infrasound, auditory sound, and ultrasound. In these implementations, nipple may comprise a material that is substantially transparent to acoustic vibrations. In other implementations, narrower frequency domains may be employed (e.g., an implementation may be limited to one of infrasound, auditory sound, or ultrasound) and nipple 602 may comprise a material that is transparent to these ranges. For instance, an implementation may utilize frequencies between 100 Hz-1 kHz (e.g., between 100-700 Hz, 400-600 Hz, etc.). In some implementations, the nipple may be shaped (e.g., molded) to provide a shape conducive to transmission and reception of sound waves, such as with particular concave or convex surfaces, etc.

In some embodiments, including when adults desire respiratory monitoring, the intraoral device 601 (e.g., a mouthpiece) can include a mouthpiece (e.g., a night guard, a dental aligner that can be suitable for sleep), a tray, an adult pacifier, etc. In other cases, non-contact approaches can provide respiratory monitoring that lack a mouthpiece. For example, the system 600 can include a computing device including the one or more transducers (e.g., a smartphone) to detect active, passive, etc., sounds from a subject and analyze the sounds accordingly. In this case, the computing device can be placed near the subject as the subject sleeps (e.g., on a nightstand) and can monitor the subject while the subject sleeps. In other cases, the one or more transducers can be mounted (e.g., removably coupled) near the subject's nose (e.g., with a clip) to transmit sounds into the respiratory system and receive sound signals therefrom. In yet other cases, the one or more transducers can be mounted near a subject's mouth or nose (e.g., on a shirt, sleeping garment, etc., using a clip).

In some implementations, device 601 may comprise a sonic transmitter (e.g., an acoustic transducer, an acoustic transmitter, a speaker, etc.) and may comprise a sonic receiver (e.g., an acoustic transducer, an acoustic receiver, a microphone). The sonic transmitter can be disposed within the nipple to transmit an emitted sound signal to a respiratory system in an active sound mode. Similarly, the sonic receiver (e.g., an acoustic transducer, an acoustic receiver, a microphone) can be disposed within the nipple to receive a reflected sound signal from the respiratory system in the active sound mode and to receive a passive breathing sound signal in a passive sound mode. For example, a sonic transmitter may comprise a first sonic transducer 604 disposed within the nipple and coupled to circuitry 607 via a flexible connector 617 (e.g., flexible PCB, ribbon connector, etc.). Similarly, a sonic receiver may comprise a second transducer 609 coupled to circuitry 607 via connector 617. In various implementations, transducers 604, 609 may comprise any suitable devices for transmitting and receiving sound waves, such a speaker and microphone or a plurality of microphones (e.g., a 2D array of microphones). In some cases, the plurality of microphones can be used for sound phase detection. In some cases, the transducer 604, 609 may comprise piezoelectric, capacitive, micro-electromechanical system (MEMS), electret speakers/microphone (e.g., an electret condenser microphone (ECM)), etc. In various implementations, transducers 604, 609 may be suitably sized to be disposed within a device 601 such as a pacifier. For instance, transducers 604, 609 may have dimensions of under 5 mm×5 mm. In various examples, transducers 604, 609 may provide transmission/capture of frequencies as discussed herein (e.g., between about 10 Hz to 40 kHz). For example, the transmission sound (e.g., from an acoustic transducer that emits sound in the respiratory system) can have a frequency (e.g., a fundamental frequency) that is between about 10 Hz to 40 kHz, less than 1 kHz, less than 900 Hz, less than 800 Hz, less than 700 Hz, less than 600 Hz, less than 500 Hz, less than 400 Hz, less than 200 Hz, less than 100 Hz. In some cases, the frequency can be in a range between about 100 Hz and 700 Hz. In some cases, the frequency can be about 400 Hz, which, as described above, can provide lower losses. As used herein, the term “about” when used with respect to a reference value can refer to variations from the reference value of ±15% or less (e.g., ±10%, ±5%, etc.), inclusive of the endpoints of the range.

In some cases, the system 600 can include a first acoustic transducer for emitting sound signals into the respiratory system of the subject (e.g., a single, sonic transducer, a single ultrasonic transducer, etc.) and a plurality of acoustic transducers for receiving sound signals from the respiratory system of the subject. For example, the plurality of acoustic transducers can be in an array (e.g., a single array, one-dimensional array, or a two-dimensional array). In some cases, an array of 2D acoustic transducers can be a two-by-two array (i.e., a 2×2 array), a three-by-three array (i.e., a 3×3 array), a four-by-four array (i.e., a 4×4 array), etc. By having a plurality of acoustic transducers, and more specifically, an array of acoustic transducers, can help determine phase information from the reflected sound signals (e.g., received from the respiratory system of the subject).

In further implementations, a sonic transmitter or sonic receiver may comprise a waveguide disposed within the nipple 602 to couple sound to transducers disposed on a system circuit board 607. For example, such a waveguide may include structures such as a conical air cavity 610 or a piezoelectric gel waveguide 611. Such waveguides may be configured to transmit and receive particular frequencies or frequency bands or may be adaptable to transmit and receive selected frequencies/frequency bands. For instance, a piezoelectric gel waveguide 611 may be operated to change its acoustic properties via piezoelectric expansion/contraction or other change of material properties to provide sound amplification (e.g., an acoustic gain material) at desired frequencies. Of course, further implementation may provide any sound-air interface to enhance the detection of lung characteristics, with varying transmitter and receiver placement, acoustic pathway design, and algorithm development for interpreting sound wave patterns, etc.

In some configurations, each transducer 604, 609 can be positioned within a cavity, recess, or hole, in the mouthpiece 602 (e.g., the conical air cavity 610). In some cases, the hole, cavity, recess, etc., of the mouthpiece 602 does not extend entirely through the mouthpiece 602 (e.g., through a free end thereof). For example, when the mouthpiece 602 is a nipple, the cavity, recess, etc., does not extend through to the outer surface of the nipple. In this way, bodily fluids of the neonate do not flow into the hole and damage the electrical components of the system 600. In some cases, one end of the connector 617 can be coupled to a printed circuit board (PCB) or other substate that supports or contains the electrical components of the system 600 (e.g., amplifiers, power sources, communication modules, power supplies, controllers, processors, sensors, etc.) and the opposing end of the connector 617 can be coupled to the transducers 604, 609. In some cases, and as shown in FIG. 6A, the transducers 604, 609 can be positioned at the same longitudinal position along the connector 617. However, in other cases, the transducers 604, 609 can be positioned at different longitudinal positions along the connector 617. For example, the transducer 604 can be positioned in front of the transducer 609, such that the transducer 609 is closer to the base of the connector 617 than the transducer 604. In this way, the transducer 609 can avoid blocking the transmission of sound waves emitted by the transducer 604. In some configurations, the system 600 may lack a transmitter, or acoustic transducer that emits sound, such as when the system 600 performs only passive sensing. In some cases, the system 600 can include an acoustic transducer that emits sound towards the subject (e.g., into the respiratory system thereof) and also receives sound from the respiratory system of the subject (e.g., for conversion into appropriate electrical signals, including for example, using an analog to digital converter (ADC)).

In some implementations, device 601 may further include sensors 603 disposed within the mouthpiece 602 or otherwise located to provide various intraoral sensor modalities. For instance, sensors 603 may include motion sensors such as individual or combined accelerometers, gyroscopic sensors, magnetometers, etc. (e.g., inertial measurement units (IMUs)). As further examples, sensors 603 may comprise oximeters, temperature sensors, pulse monitors, etc.

In some examples, device 601 may include neural network circuitry 607 to execute a neural network to generate an output indicative of a health condition of the respiratory system. For example, neural network circuitry 607 may comprise a processor 607 and a non-transitory computer readable medium 616 storing instructions executable by the processor to execute the neural network. For example, processor 607 may comprise a general-purpose processor, an accelerator, a neural network/AI processor, a system-on-a-chip (SoC), etc. For example, medium 616 may comprise a flash memory. In some examples, medium 616 may comprise a removable storage medium, such as a Secure Digital (SD) card or other insertable storage device. As another example, neural network circuitry 607 may other hardware logic to execute the neural network, such as neuromorphic hardware, an application specific integrated circuit (ASIC), field-programmable gate array (FPGA), etc.

In some cases, one or more portions of (or the entire) machine learning model (e.g., a trained model) can be implemented on a computing device, such as, a smartphone, a computer, etc. In this case, the system 600 can transmit sound signals, data, etc., to the computing device for further analysis. In other cases, the system 600 (e.g., the mouthpiece 601) can run one or more portions of (or the entire) machine learning model and outputs (e.g., results, warnings, notifications, respiratory parameters, respiratory condition diagnoses, etc.) can be transmitted (e.g., periodically) to the computing device (e.g., smartphone). In this way, the system 600 performs as an edge AI (e.g., where the processing is done on the device and results are communicated to the computing device, such as via a Bluetooth^{®}communication protocol) and avoids the need for an internet connection. This can provide better preservation of data as well as confidentiality and security (e.g., of patient health information).

In some examples, the neural network may comprise a neural network as described with respect to FIG. 3 or FIG. 4 to generate the output based on a reflected or passive sound signal captured by microphone transducer 609. In further examples, the neural network may include inputs to receive various sensor data from sensors 603. For instance, sensor data may provide contextual data or features to support attention mechanisms, Bayesian inferences, etc. In some implementations, circuitry 607 may comprise audio processing circuitry (e.g., as described with respect to preprocessing pipelines 302, 416), and may condition signals such as via amplitude, phase, frequency shifts, time of flight, etc. to extract the desired data while sweeping the sound frequency and other algorithms for interpreting sound wave patterns.

In some examples, device 601 may include a wireless transceiver (e.g., a Bluetooth Low Energy (BLE) or other body/personal area network transceiver, a WiFi transceiver, etc.) and control circuitry 606. For instance, circuitry 606 may comprise circuitry to control operation of neural network circuitry 607, to collect and transmit sensor data, to control frequency modulations, operational modes, to receive commands, etc. As a particular example, circuitry 606 may comprise logic or programming to enter an active sound mode responsive to a triggering condition, such as a detected possible health condition via a passive sound mode, an elapsed time since a previous active mode operation, a received command, etc. Device 601 may include further componentry to support its functions, such as a power source 608 (e.g., a battery, such as a coin battery, which may fit better within a housing 605 that can be circular, a rechargeable battery, etc.). In some implementations, some or all of circuitry and components 606, 607, 608, 603 may be disposed within a housing 605 (e.g., a pacifier handle). For example, the housing 605 can be coupled to the mouthpiece 602, and can be sandwiched together to seal the components 606, 607, 608, 603 within the housing 605 and the mouthpiece 602 (e.g., hermetically sealed). In this way, bodily fluids, such as saliva do not interfere with the electrical components of the system 600. In some cases, the mouthpiece 602 and the housing 605 can be removably coupled together, such as by snap and fit connections, hook and loop fasteners, threaded engagement, magnets, etc. In this way, when sensing or detecting is desired to be stopped, such as when the neonate newborn, infant, etc., is awake, the mouthpiece 602 and the housing 605 can be decoupled and replaced. Alternatively, in some cases, the housing 605 can be coupled to the other components 606, 607, 608, 606, such that the mouthpiece 602 can be decoupled (and thrown away, cleaned, or sanitized) and recoupled (or a different new mouthpiece 602 is recoupled) when the system 600 is desired to be used again. In some cases, such as when the power source is a rechargeable battery, the rechargeable battery can be recharged when not in use (and with the mouthpiece 602 decoupled therefrom).

In some examples, a trained neural network 615 may be instantiated in neural network circuitry 607. In various implementations, device 601 may be provided with the trained network 615 (e.g., also described as a trained model) in any suitable manner, such as via programming during device manufacture, via wireless transmission using a wireless transceiver (e.g., a BLE interface), via insertion of a suitably programmed removable storage medium 616, etc. In some examples, a common pre-trained network 615 may be instantiated in multiple different devices 601. For instance, a common pre-trained network 615 for multiple subject classes may be used in different devices (e.g., a single model for neonates, children, adults, etc.). As another example, a common pre-trained network 615 may be used for multiple different sound domain modalities (e.g., a single model for infrasound, auditory sound, and ultrasound). In further implementations, different pre-trained networks 615 may be deployed according to various conditions, such as different networks 615 for different subject classes (e.g., different networks for premature neonates, full-term neonates, children, adults, etc.), for different sound frequency ranges (e.g., different networks for infrasound implementations, auditory sound implementations, ultrasound implementations, etc.), combinations thereof, etc. In still further examples, subject-specific networks 615 may be trained and deployed (e.g., via additional training applied to a generic pre-trained network, or a network trained on a specific subject's data).

In some implementations, system 600 may comprise other devices 618 that may connect to device 601. For instance, a device 618 such as a smartphone, laptop, or other computer may connect to device 601 via a wireless interface. Device 618 may interface with device 601 to receive data, such as sensor data, respiratory function data, event data, etc. For example, device 618 may receive poll device 601 to receive a record of a current or historical average respiratory profile, a current or historical respiratory health status, a current or historical instantaneous respiratory profile. As another example, device 618 may interface with device 601 to transmit data, such as commands, trained neural networks, etc. In some examples, device 618 may provide a connection 619 (e.g., via the Internet, local area network, etc.) to a clinician or other health care provider 620. For example, device 618 may support a home or other remote deployment of a device 601 to provide respiratory health monitoring outside of a direct clinical setting.

### Neural Network Implementation

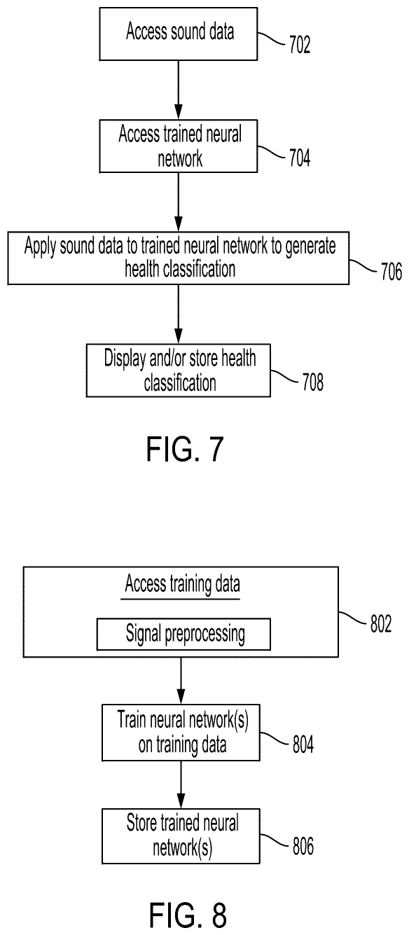

Referring now to FIG. 7, a flowchart is illustrated as setting forth the steps of an example method for respiratory function characterization using a suitably trained neural network or other machine learning model, such as, for example, the neural networks described with respect to FIGS. 3, 4. As will be described, the neural network or other machine learning model takes sound data (e.g., passive sound signals or reflected sound signals) as input data and generates respiratory health data as output data.

### Accessing Data

The method includes accessing respiratory sound data with a computer system, as indicated at step 702. Accessing the respiratory sound data may include retrieving such data from a memory or other suitable data storage device or medium. Additionally or alternatively, accessing the respiratory sound data may include acquiring such data with an intraoral sound transmission/reception system and transferring or otherwise communicating the data to the computer system, which may be a part of a wearable medical device that includes the intraoral sound transmission/reception system. As described above, the respiratory sound data are generally passive sound signals recorded while a subject is breathing or reflected sound signals recorded while a subject is breathing in the presence of a sound stimulus, and in some instances may be audio spectrogram data.

### Accessing Trained Neural Network

A trained neural network (or other suitable machine learning model) is then accessed with the computer system, as indicated at step 704. In general, the neural network is trained, or has been trained, on training data in order to evaluate respiratory health, such as a health classification or respiratory model (e.g., a “digital twin” of a subject's respiratory system). This evaluation is achieved, in part, by the neural network (or other machine learning model) being trained via a breathing sound dataset. For example, the training dataset may comprise breathing audio signals captured from the intraoral active and passive sound sensing approaches, which may be annotated with the ground truth measures.

The trained neural network can include a neural network with any suitable neural network architecture for generating respiratory health data. As one non-limiting example, the trained neural network may include a convolutional neural network, such as a convolutional neural network comprising feature extraction subnetworks for passive and active sound signal analysis, and feature generation subnetworks for active sound signal analysis. The trained neural network may in some instances have multiple inputs (e.g., corresponding to sound signals and other sensor data).

Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed. In some examples, accessing the trained neural network may comprise operating a neural network instantiated on a mobile device, such as described with respect to FIG. 6.

### Applying Data to the Neural Network

The sound signal data are then input to the trained neural network, generating output as respiratory health data, as indicated at step 706. For example, the respiratory health data may comprise a classification of health status (e.g., a binary healthy/unhealthy status indicator) or a regression of respiratory conditions/characteristics. As an example, the sound signal data may include raw sound signals recorded from a subject's respiratory system. As another example, the sound signal data may comprise spectrogram data constructed from recorded raw sound signals. In some cases, a health classification can be a classification for a particular respiratory disease state, respiratory condition, lung disease state, lung condition, etc. For example, the specific respiratory disease state or respiratory condition can be COPD, shortness of breath, airway blockage disease, diabetes, BPD, RDS, pneumonia, pulmonary edema, tracheal stenosis, asthma, bronchitis, apnea (including sleep apnea), PRD, etc. In some configurations, the respiratory health data outputted at step 706 can include a respiratory parameter, such as, for example, FVC, FEV1/FVC, SPO2, HR, BR, VO2Max, etc. For example, the respiratory parameter can include a type (e.g., BR) and a value (e.g., 12 breaths per minute).

In some cases, the step 706 can include providing a confidence score for each respiratory condition. For example, this can include providing a confidence score of 85% for the subject having an airway blockage disease, a confidence score of 65% for the subject having a pulmonary edema, and a confidence score of 95% for the subject having bronchopulmonary dysplasia. Accordingly, the step 706 can provide a multi-class classification of different pulmonary conditions.

### Display And/or Store Output

The respiratory health data generated by inputting the sound signal data to the trained neural network(s) can then be provided to a user, stored for later use or further processing, or both, as indicated at step 708. For example, the respiratory health data may be stored on a wearable device memory (e.g., memory 616 of FIG. 6B), may be transmitted to an attached device (e.g., via a BLE connection to an attached device 618), uploaded to a server, such as a clinical computer system (e.g., a server at a clinic 620), displayed on the wearable device (e.g., via an indicator light to indicate a health status output), etc.

### Neural Network Training

Referring now to FIG. 8, a flowchart is illustrated as setting forth the steps of an example method for training one or more neural networks (or other suitable machine learning models) on training data, such that the one or more neural networks are trained to receive sound signal data as input data in order to generate respiratory health data as output data. For example, in some implementations, neural network training may be performed on a computer system (e.g., system 615) prior to being instantiated on a wearable device (e.g., device 601).

In general, the neural network(s) can implement any number of different neural network architectures. For instance, the neural network(s) could implement a convolutional neural network, a residual neural network, or the like. Alternatively, the neural network(s) could be replaced with other suitable machine learning or artificial intelligence algorithms, such as those based on supervised learning, unsupervised learning, deep learning, ensemble learning, dimensionality reduction, and so on.

### Accessing Training Data

The method includes accessing training data with a computer system, as indicated at step 802. In general, the training data can include breathing sound data with ground truth annotations generated from input sound signal data. Additionally or alternatively, the accessed training data can include sound data received from an example database. Accessing the training data may include retrieving such data from a memory or other suitable data storage device or medium. Alternatively, accessing the training data may include acquiring such data with an intraoral sound recordation device and transferring or otherwise communicating the data to the computer system.

The method can include assembling training data from sound signal data using a computer system. This step may include assembling the sound signal data into an appropriate data structure on which the neural network or other machine learning model can be trained. Assembling the training data may include annotating sound data. For instance, assembling the training data may include recording or obtaining sound signals, annotating the sound signals with known respiratory health ground truth values, preprocessing sound data, and the like.

### Training the Neural Network

One or more neural networks (or other suitable machine learning models) are trained on the training data, as indicated at step 804. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as respiratory health data. The artificial neural network then compares the generated output with a ground truth value of the training example in order to evaluate the quality of the respiratory health data. For instance, the respiratory health data can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the artificial neural network is configured to learn a general rule or model that maps the inputs to the outputs based on the provided example input-output pairs.

### Storing Neural Network(s) for Later Use

The one or more trained neural networks are then stored for later use, as indicated at step 806. Storing the neural network(s) may include storing network parameters (e.g., weights, biases, or both), which have been computed or otherwise estimated by training the neural network(s) on the training data. For example, storing the neural network may include instantiating the neural network on a wearable device, such by programming a neuromorphic computer or storing neural network parameters in a wearable device storage system, controller, etc. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

### Computer Systems

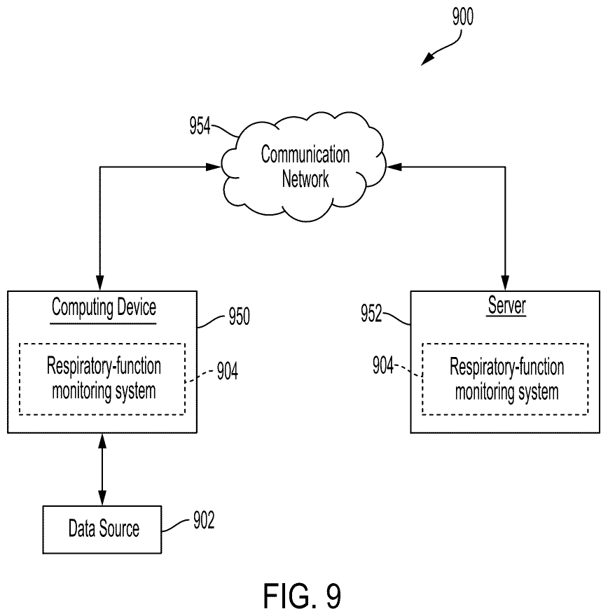

FIG. 9 shows an example of a system 900 for evaluating respiratory health in accordance with some embodiments described in the present disclosure. As shown in FIG. 9, a computing device 950 can receive one or more types of data (e.g., sound signal data, including raw sound signals and/or spectrogram data) from data source 902. For example, computing device 950 may comprise a wearable device and data source 902 may comprise a microphone or other source of sound signal data. In some embodiments, computing device 950 can execute at least a portion of respiratory-function monitoring system 904 to generate health data (e.g., respiratory health data) from data received from the data source 902.

Additionally or alternatively, in some embodiments, the computing device 950 can communicate information about data received from the data source 902 to a server 952 over a communication network 954, which can execute at least a portion of the respiratory-function monitoring system 904. For example, server 952 may comprise a mobile device connected to a wearable device (e.g., mobile device 618) or a remote server in a clinic. In such embodiments, the server 952 can return information to the computing device 950 (and/or any other suitable computing device, such as a mobile device 618) indicative of an output of the respiratory-function monitoring system 904.

In some embodiments, computing device 950 and/or server 952 can be any suitable computing device or combination of devices, such as a wearable computer, a smartphone, a desktop computer, a laptop computer, a tablet computer, a server computer, a virtual machine being executed by a physical computing device, and so on.

In some embodiments, data source 902 can be any suitable source of data (e.g., recorded sound data, spectrogram data reconstructed from recorded sound data, etc.), such as a sound recording transducer, another computing device (e.g., a server storing sound data, spectrogram data, etc.), and so on. In some embodiments, data source 902 can be local to computing device 950. For example, data source 902 can be incorporated with computing device 950 (e.g., computing device 950 can be configured as part of a device for measuring, recording, estimating, acquiring, or otherwise collecting or storing data). As another example, data source 902 can be connected to computing device 950 by a cable, a direct wireless link, and so on. Additionally or alternatively, in some embodiments, data source 902 can be located locally and/or remotely from computing device 950, and can communicate data to computing device 950 (and/or server 952) via a communication network (e.g., communication network 954).

In some embodiments, communication network 954 can be any suitable communication network or combination of communication networks. For example, communication network 954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 954 can be a local area network, a wide area network, a public network (e.g., the Internet), a private or semi-private network (e.g., a corporate or university intranet), any other suitable type of network, or any suitable combination of networks. Communications links shown in FIG. 9 can each be any suitable communications link or combination of communications links, such as wired links, fiber optic links, Wi-Fi links, Bluetooth links, cellular links, and so on.

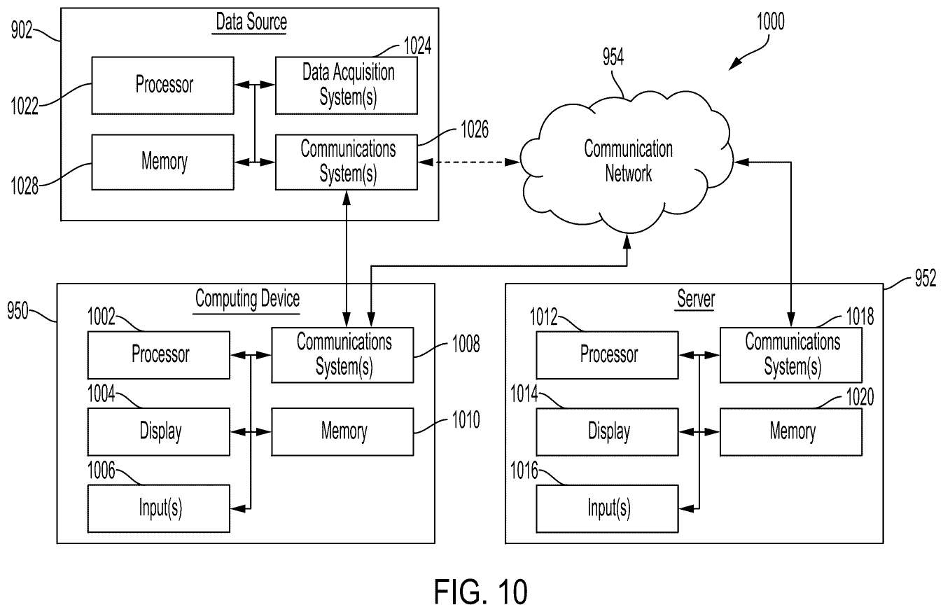

Referring now to FIG. 10, an example of hardware 1000 that can be used to implement data source 902, computing device 950, and server 952 in accordance with some embodiments of the systems and methods described in the present disclosure is shown.

As shown in FIG. 10, in some embodiments, computing device 950 can include a processor 1002, a display 1004, one or more inputs 1006, one or more communication systems 1008, and/or memory 1010. In some embodiments, processor 1002 can be any suitable hardware processor or combination of processors, such as a central processing unit (CPU), a graphics processing unit (GPU), and so on. In some embodiments, display 1004 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 1006 can include any suitable input devices and/or sensors that can be used to receive user input, such as a keyboard, a mouse, a touchscreen, a microphone, and so on.

In some embodiments, communications systems 1008 can include any suitable hardware, firmware, and/or software for communicating information over communication network 954 and/or any other suitable communication networks. For example, communications systems 1008 can include one or more transceivers, one or more communication chips and/or chip sets, and so on. In a more particular example, communications systems 1008 can include hardware, firmware, and/or software that can be used to establish a Wi-Fi connection, a Bluetooth connection, a cellular connection, an Ethernet connection, and so on.

In some embodiments, memory 1010 can include any suitable storage device or devices that can be used to store instructions, values, data, or the like, that can be used, for example, by processor 1002 to present content using display 1004, to communicate with server 952 via communications system(s) 1008, and so on. Memory 1010 can include any suitable volatile memory, non-volatile memory, storage, or any suitable combination thereof. For example, memory 1010 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 1010 can have encoded thereon, or otherwise stored therein, a computer program for controlling operation of computing device 950. In such embodiments, processor 1002 can execute at least a portion of the computer program to present content (e.g., images, user interfaces, graphics, tables), receive content from server 952, transmit information to server 952, and so on. For example, the processor 1002 and the memory 1010 can be configured to perform the methods described herein (e.g., the method of FIG. 7, the method of FIG. 8).

In some embodiments, server 952 can include a processor 1012, a display 1014, one or more inputs 1016, one or more communications systems 1018, and/or memory 1020. In some embodiments, processor 1012 can be any suitable hardware processor or combination of processors, such as a CPU, a GPU, and so on. In some embodiments, display 1014 can include any suitable display devices, such as an LCD screen, LED display, OLED display, electrophoretic display, a computer monitor, a touchscreen, a television, and so on. In some embodiments, inputs 1016 can include any suitable input devices and/or sensors that can be used to receive user input, such as a keyboard, a mouse, a touchscreen, a microphone, and so on.

In some embodiments, communications systems 1018 can include any suitable hardware, firmware, and/or software for communicating information over communication network 954 and/or any other suitable communication networks. For example, communications systems 1018 can include one or more transceivers, one or more communication chips and/or chip sets, and so on. In a more particular example, communications systems 1018 can include hardware, firmware, and/or software that can be used to establish a Wi-Fi connection, a Bluetooth connection, a cellular connection, an Ethernet connection, and so on.

In some embodiments, memory 1020 can include any suitable storage device or devices that can be used to store instructions, values, data, or the like, that can be used, for example, by processor 1012 to present content using display 1014, to communicate with one or more computing devices 950, and so on. Memory 1020 can include any suitable volatile memory, non-volatile memory, storage, or any suitable combination thereof. For example, memory 1020 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1020 can have encoded thereon a server program for controlling operation of server 952. In such embodiments, processor 1012 can execute at least a portion of the server program to transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone), and so on.

In some embodiments, the server 952 is configured to perform the methods described in the present disclosure. For example, the processor 1012 and memory 1020 can be configured to perform the methods described herein (e.g., the method of FIG. 7, the method of FIG. 8).

In some embodiments, data source 902 can include a processor 1022, one or more data acquisition systems 1024 (e.g., sensors), one or more communications systems 1026, and/or memory 1028. In some embodiments, processor 1022 can be any suitable hardware processor or combination of processors, such as a CPU, a GPU, and so on. In some embodiments, the one or more data acquisition systems 1024 comprise intraoral sensors to acquire data, such as sound recordings, or processing systems to produce spectrograms. Additionally or alternatively, in some embodiments, the one or more data acquisition systems 1024 can include any suitable hardware, firmware, and/or software for coupling to and/or controlling operations of an intraoral sound recording system. In some embodiments, one or more portions of the data acquisition system(s) 1024 can be removable and/or replaceable.

In some embodiments, communications systems 1026 can include any suitable hardware, firmware, and/or software for communicating information to computing device 950 (and, in some embodiments, over communication network 954 and/or any other suitable communication networks). For example, communications systems 1026 can include one or more transceivers, one or more communication chips and/or chip sets, and so on. In a more particular example, communications systems 1026 can include hardware, firmware, and/or software that can be used to establish a wired connection using any suitable port and/or communication standard (e.g., DVI video, USB, RS-232, etc.), Wi-Fi connection, a Bluetooth connection, a cellular connection, an Ethernet connection, and so on.

In some embodiments, memory 1028 can include any suitable storage device or devices that can be used to store instructions, values, data, or the like, that can be used, for example, by processor 1022 to control the one or more data acquisition systems 1024, and/or receive data from the one or more data acquisition systems 1024; to generate images from data; present content (e.g., data, images, a user interface) using a display; communicate with one or more computing devices 950; and so on. Memory 1028 can include any suitable volatile memory, non-volatile memory, storage, or any suitable combination thereof. For example, memory 1028 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1028 can have encoded thereon, or otherwise stored therein, a program for controlling operation of data source 902. In such embodiments, processor 1022 can execute at least a portion of the program to generate images, transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

1. An apparatus, comprising:
a mouthpiece;
a sonic transmitter disposed within the mouthpiece to transmit an emitted sound signal to a respiratory system;
a sonic receiver disposed within the mouthpiece to receive a reflected sound signal from the respiratory system; and
neural network circuitry to execute a neural network to generate an output indicative of a health condition of the respiratory system based on the reflected sound signal.

2. The apparatus of claim 1, further comprising transform circuitry to generate spectrum data based on the reflected sound signal, and wherein the neural network is to generate the output indicative of the health condition based on the spectrum data.

3. The apparatus of claim 1, wherein:
the reflected sound signal comprises an active reflected sound signal; and
the sonic receiver is to receive a passive reflected sound signal from the respiratory system.

4. The apparatus of claim 3, wherein the neural network comprises a feature extraction subnetwork to process the active reflected sound signal and the passive reflected sound signal.

5. The apparatus of claim 4, wherein at least one of:
the feature extraction subnetwork comprises a dilated convolution layer; or
the neural network further comprises a feature construction subnetwork to process an output of the feature extraction subnetwork corresponding to the active reflected sound signal.

6. (canceled)

7. The apparatus of claim 1, wherein the neural network further comprises a feature construction subnetwork to process an output of the feature extraction subnetwork corresponding to the active reflected sound signal; and
wherein at least one of:
the feature construction subnetwork comprises a data removal subnetwork, a data retrieval subnetwork coupled to the data removal subnetwork, and a feature generation subnetwork coupled to the data retrieval subnetwork; or
the neural network further comprises a classifier to output a binary health classification, and a regression output to output a predicted respiratory condition.

8. The apparatus of claim 1, wherein the neural network further comprises a plurality of subnetwork layers, each respective subnetwork layer comprising:
a respective feature extraction subnetwork to process data corresponding to the active reflected sound signal and the passive reflected sound signal; and
a respective feature generation subnetwork to process data corresponding to the active reflected sound signal.

9-14. (canceled)

15. The apparatus of claim 1, wherein:
the sonic transmitter comprises a waveguide disposed within the mouthpiece coupled to a first sonic transducer; and
the sonic receiver comprises a second sonic transducer coupled to the waveguide;
the waveguide includes a piezoelectric gel waveguide or a comprises an air cavity waveguide; and further comprising a handle housing comprising the first sonic transducer, the second sonic transducer, and the neural network circuitry.

16-18. (canceled)

19. The apparatus of claim 1, wherein:
the sonic transmitter comprises a first sonic transducer disposed within the mouthpiece; and
the sonic receiver comprises a second sonic transducer disposed within the mouthpiece.

20. The apparatus of claim 1, further comprising a pulse oximeter, an inertial measurement unit, or a temperature sensor.

21. (canceled)

22. The apparatus of claim 1, further comprising:
a housing for a smart pacifier comprising the neural network circuitry, wherein the mouthpiece comprises a nipple for the smart pacifier.

23. An apparatus, comprising:
a mouthpiece;
one or more acoustic transducers coupled to the mouthpiece; and
a processor in communication with the one or more acoustic transducers, the processor configured to:
receive, from the one or more acoustic transducers, a sound signal from a respiratory system;
provide the sound signal to a trained model that identifies respiratory health conditions; and
receive, from the trained model, an output indicative of a respiratory health condition.

24. The apparatus of claim 23, wherein the sound signal is a receiving sound signal; and
wherein the one or more acoustic transducers are configured to emit a transmission sound signal into the respiratory system, the transmission sound signal being a stimulus sound signal that produces the receiving sound signal in response to the stimulus sound signal.

25. The apparatus of claim 24, wherein the transmission sound signal has a frequency that is about 400 Hz.

26. (canceled)

27. The apparatus of claim 24, wherein the one or more transducers include:
a first acoustic transducer configured to receive the receiving sound signal; and
a second acoustic transducer configured to transmit the transmission sound signal.

28-33. (canceled)

34. The apparatus of claim 23, wherein the respiratory health condition is a at least one of chronic obstructive pulmonary disease (COPD), shortness of breath, airway blockage disease, diabetes, bronchopulmonary dysplasia (BPD), respiratory distress syndrome (RDS), pneumonia, pulmonary edema, tracheal stenosis, asthma, bronchitis, apnea, sleep apnea, or prematurity respiratory disease (PRD).

35-36. (canceled)

37. A method comprising:
receiving, using one or more computing devices, a sound signal, the sound signal being from a respiratory system of a subject;
providing, using the one or more computing devices, the sound signal to a trained model that identifies a respiratory health condition or a respiratory parameter; and
receiving, using the one or more computing devices, an output from the trained model indicative of the respiratory health condition or the respiratory parameter.

38. The method of claim 37, wherein the sound signal is a receiving sound signal and further comprising transmitting, using the one or more computing devices, a transmission sound signal into the respiratory system of the subject; and
wherein the transmission sound signal is a stimulus sound signal that produces the receiving sound signal in response to the stimulus sound signal.

39. The method of claim 38, wherein the receiving sound signal is a first receiving sound signal and further comprising receiving, using the one or more computing devices, a second receiving sound signal, the second receiving sound signal being from a respiratory system of a subject;
wherein the first receiving sound signal is an active sound signal from the stimulus sound signal; and
wherein the second sound signal is a passive sound signal acquired in the absence of a stimulus sound signal provided to the respiratory system.

40. The method of claim 39, further comprising providing, using the one or more computing devices, the active sound signal and the passive sound signal to a trained model that identifies the respiratory health condition or the respiratory parameter.

41-43. (canceled)
